(19) United States
(12) Patent Application Publication
(10) Pub. No.: US 2026/0144596 A1
(43) Pub. Date: May 28, 2026

(54) SYSTEM AND METHOD OF USING SURGICAL NAVIGATION AND PLANNING TOOLS

(71) Applicant: PERSPECTIVE TECHNOLOGIES, LLC, Orlando, FL (US)

(72) Inventor: Maahir Haque, Orlando, FL (US)

(21) Appl. No.: 19/122,186

(22) PCT Filed: Oct. 20, 2023
(86) PCT No.: PCT/US2023/077367

§ 371 Date: Apr. 17, 2025

Related U.S. Application Data
(60) Provisional application No. 63/380,497, filed on Oct. 21, 2022.

(51) Int. Cl. A61B 34/20; A61B 34/00; A61B 90/00

(52) U.S. Cl. CPC A61B 34/20; A61B 34/76; A61B 90/03; A61B 2560/0223

(57) ABSTRACT

A system includes a controller configured to receive three dimensional (3D) imaging input and feedback and generate output, the 3D imaging input including real-time or pre-processed data of a magnetic resonance imaging (MRI)/computerized tomography (CT) scan, the controller interfacing with a stereotactic robot.

### CROSS-REFERENCE TO RELATED APPLICATION

The present application claims priority benefit of U.S. Provisional Application No. 63/380,497, filed Oct. 21, 2022, which is herein incorporated by reference in its entirety.

### BACKGROUND OF THE INVENTION

The present invention generally relates to medical surgeries, and in particular, to a system and method of using surgical navigational and planning tools.

In general, during robotic-assisted surgeries, a surgeon physically interacts with a control device, which communicates with a robotic surgical device that manually performs the desired surgical task. With such a robotic surgical device, however, there may be a physical disconnect between the surgeon and the patient's body which can present potential challenges as well as benefits.

One of the disadvantages of this disconnect is the loss of tactile feedback provided by physical manipulation of tissues. In some instances, robotic surgical devices may be programmed to provide feedback through the control device for overcoming this lack of feedback.

One of the advantages of this disconnect is the ability to insulate a surgeon from the unintended consequences of certain surgical instruments. For example, burr or harmonic scalpel devices may produce mechanical vibrations during use whereby such vibrations can be quite significant. These vibrations, nonetheless, may mask useful feedback transmitted from the likes of tissues or bones. In some instances, when navigating tissues of varying densities adjacent critical structures such as blood vessels, nerve structures, or organs, there may be a need to insulate such critical structures from these detrimental forces.

As such, there exists a need for surgical navigation and planning tools, systems and methods thereof that can improve these shortcomings.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In an aspect, the invention features a method of using surgical navigation and planning tools, the method including obtaining three dimensional (3D) imaging data of a patient by an imaging technique, concurrently with obtaining the 3D imaging data, obtaining stereotactic information from an intraoperative tool tracked by a surgical robotic device and associated robotic system, processing the 3D imaging data and the stereotactic information, integrating the 3D imaging data and the stereotactic information and interpreting volumetric data to assess a patient's local tissue density at the tip of a directional vector of the surgical tool, and providing haptic feedback during the surgical procedure.

In another aspect, the invention features system including a controller configured to receive three dimensional (3D) imaging input and feedback and generate output, the 3D imaging input including real-time or pre-processed data of a magnetic resonance imaging (MRI)/computerized tomography (CT) scan, the controller interfacing with a stereotactic robot.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF DRAWINGS

These and other aspects will now be described in detail with reference to the accompanying drawings, wherein:

FIG. 1A illustrates an exemplary method of using surgical navigation, planning tools and system thereof in accordance with an embodiment.

FIG. 1B illustrates an exemplary method of using surgical navigation, planning tools and system thereof in accordance with an embodiment.

FIG. 2 illustrates an exemplary surgical navigation and planning tools and system thereof according to an embodiment.

FIG. 3 illustrates an exemplary system architecture for implementing the disclosed embodiments of surgical navigation and planning tools and methods of use thereof.

FIG. 4 is a block diagram.

### DETAILED DESCRIPTION OF THE PREFERRED EMBODIMENT(S)

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

Disclosed herein are various embodiments of surgical tools, systems and methods of processing three-dimensional (3D) data and projecting such data in a specific way that can help surgeons make decisions during robotic surgery. In addition, the disclosed embodiments are able to provide haptic feedback mechanisms based on pairing image-guidance with cross-sectional imaging.

Before performing or while performing navigated surgical procedures, it is common to use a cross-sectional imaging modality to create a model in 3D space. In one embodiment, such data may be paired with intra-operative surgical device positioning to allow stereotactic navigation. In another embodiment, in addition to providing spatial information, the data from such cross-sectional imaging may be interpreted and processed to provide additional justification and utility for such scans and/or imaging techniques.

Currently, surgeons depend primarily on visual feedback to direct a robot's movements during surgical procedures with robotic surgical devices. Disclosed is an embodiment in which information from pre-or intra-operative CT or MRI scan is processed and used to provide additional haptic feedback to a surgeon, to allow more facile control of a surgical robot when navigating and manipulating tissues of varying density or physical property.

In one embodiment, the surgical tool pairs pre-operative cross-sectional imaging data with stereotactic information intraoperatively to help surgeons more safely and deftly perform surgical procedures. This information can be helpful during surgeries which involve placement of implants into bone, which involve removal of bony structures, or which generally involve manipulation of soft tissues with different radiographic densities.

In some embodiments, the surgical tools, systems and methods disclosed herein provide additional utility for the pre-operative and intra-operative cross-sectional imaging studies that are typically performed during the planning stages of a surgical procedure. In other embodiments, the surgical tools, systems and methods disclosed herein allow surgeons to more safely, precisely, and efficiently perform surgical tasks.

In some embodiments, the surgical tools, systems and methods disclosed herein also allows closer control of patient/surgical variables to optimize patient outcomes. In other embodiments, the surgical tools, systems and methods disclosed herein benefit both surgeon and patient by offering peace of mind for helping the surgeon to blindly traverse bony or soft tissue structures adjacent to delicate structures including the likes of nerves, blood vessels and other critical organs.

In some instances, the disclosed robotic surgical embodiment can process derived tissue data from CT or MRI scans and provide feedback to the surgeon indicative of these additional tissue characteristics. For example, bone or soft tissue densities may be calculated from CT scans. This calculation may be done for purposes of tissue differentiation while interpreting the CT scans. Similarly, MRI scans can also provide soft tissue differentiation that are different from those of CT scans. As an example, the ligamentum flavum of the spine can be differentiated from the thecal sac on T2-weighted MRI whereas the same structures are much harder to differentiate on CT.

In one embodiment, after registering for stereotactic navigation off a CT scan, robotic surgical embodiments according to the present disclosure can process MRI data from the same patient and provide feedback to the surgeon indicative of the additional tissue characteristics.

In one embodiment, a method of using a system of surgical navigation and planning tools according to one embodiment includes the steps of obtaining 3D imaging data from at least one of pre-operative or intra-operative procedure, concurrently, obtaining location data from an intra-operative tool, and integrating the 3D imaging data and the location data to assess tissue density.

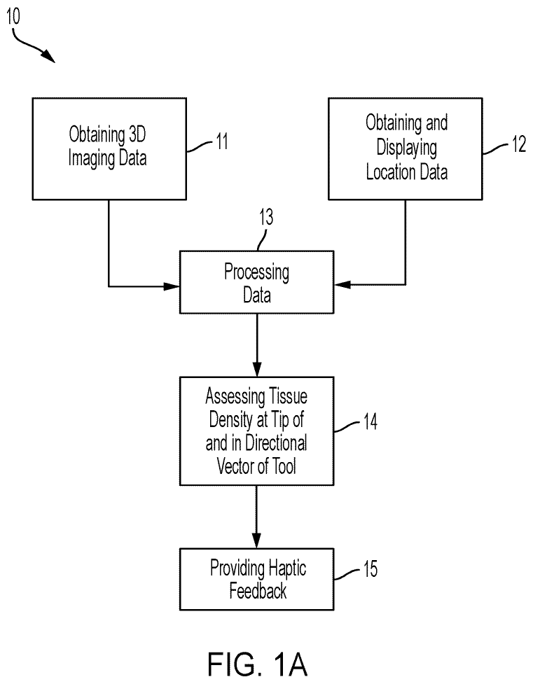

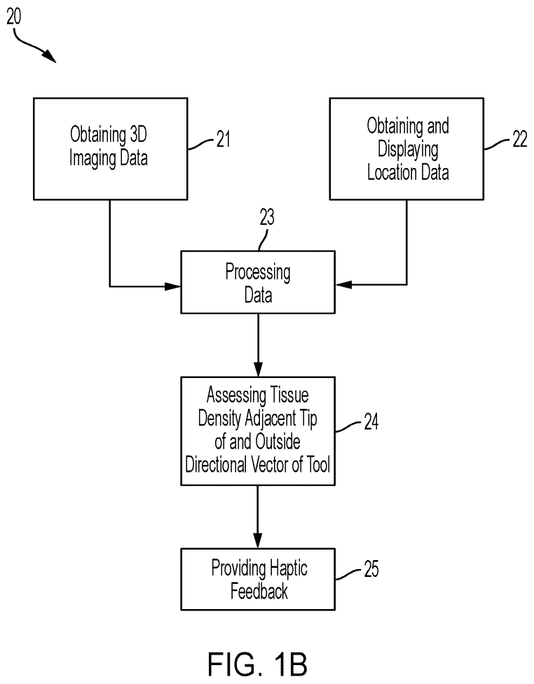

Reference is made to FIG. 1A illustrating a method 10 of using surgical navigation, planning tools and system thereof in accordance with an embodiment of the present disclosure. In this embodiment, step 11 includes obtaining 3D imaging data (e.g., volumetric data) of a patient by MRI or CT scans, or other imaging techniques. The various scanning and imaging techniques may generate two-dimensional, three-dimensional, and any other useful data, which may be acquired, processed and stored by known electronic devices and thus will not be elaborated further herein. The 3D imaging data may include pre-operative data, intra-operative data, or both.

Optionally concurrent with step 11 is step 12, which includes obtaining stereotactic information from intraoperative tool (e.g., surgical tool that physically contacts the patient during operation) tracking by surgical robotic device and associated robotic system having computer program software for execution on a computer, digital processor, microprocessor, generic or proprietary device. The robotic system can operate concurrently with the surgical robotic device and the intraoperative tool in real-time during the actual surgical procedure. In one embodiment, the surgical robotic device is able to identify, confirm, and project the location of the intraoperative tool within a patient's body onto a display in real-time that the surgeon can reference. The step of obtaining and displaying location data can be accomplished by known techniques (e.g., three-dimensional mapping with imaging cameras, navigational tools, scanners and detectors) and thus will not be elaborated further herein.

Next, in step 13, the collective data may be processed and interpreted by the same robotic system or an alternative computer processing system. The collective data include the 3D imaging data obtained during pre-operative procedures or intra-operative, or both, from step 11, as well as the 3D location data, including stereotactic information, obtained by intraoperative tool tracking, from step 12.

Next, in step 14, the system is able to integrate the collective data and interpret the volumetric data to assess a patient's local tissue density at the tip of and in directional vector of the surgical tool. In other words, this step involves assessing the tissue density at the tip of and in directional vector of a surgical tool in preparation for presenting surgeon with feedback on such tissue density, using data collected from steps 11 and 12, and processing and interpreting the same.

In one embodiment, while the processing step 13 and the assessing step 14 are shown as separate steps and in series, it will be appreciated that these steps can be performed concurrently by the same robotic system. In another embodiment, these steps may be integrated into a single step. In yet another embodiment, these steps may be provided in real-time to the surgeon during the actual surgical procedure.

Next, in step 15, the system is able to provide haptic feedback during the actual surgical procedure. The haptic feedback may include tactile detectors and other suitable haptic elements embedded throughout the robotic system. For example, a controller (not shown) for the robotic system may “push back” before an abrupt change in tissue density is expected as the surgeon is operating on the patient, and “give way” once the tissue density changes, so the actual robotic surgical tool does not advance in uncontrollable fashion.

In one embodiment, the robotic system is able to present the surgeon with feedback on tissue density at the tip of the directional vector of a tool to help surgeon execute tasks involving tissue manipulation.

Reference is made to FIG. 1B illustrating a method 20 of using surgical navigation, planning tools and system thereof in accordance with an embodiment. In this embodiment, step 21 includes obtaining 3D imaging data (e.g., volumetric data) of a patient by MRI or CT scans, or other imaging techniques. The various scanning and imaging techniques may generate two-dimensional, three-dimensional, and any other useful data, which may be acquired, processed and stored by known electronic devices and thus will not be elaborated further herein. The 3D imaging data may include pre-operative data, intra-operative data, or both.

Optionally concurrent with step 21 is step 22, which includes obtaining stereotactic information from intraoperative tool (e.g., surgical tool that physically contacts the patient during operation) tracking by surgical robotic device and associated robotic system having computer program software for execution on a computer, digital processor, microprocessor, generic or proprietary device. The robotic system can operate concurrently with the surgical robotic device and the intraoperative tool in real-time during the actual surgical procedure. In one embodiment, the surgical robotic device is able to identify, confirm, and project the location of the intraoperative tool within a patient's body onto a display in real-time that the surgeon can reference. The step of obtaining and displaying location data can be accomplished by known techniques (e.g., three-dimensional mapping with imaging cameras, navigational tools, scanners and detectors) and thus will not be elaborated further herein.

Next, in step 23, the collective data may be processed and interpreted by the same robotic system or an alternative computer processing system. The collective data include the 3D imaging data obtained during pre-operative procedures or intra-operative, or both, from step 21, as well as the 3D location data, including stereotactic information, obtained by intraoperative tool tracking, from step 22.

Next, in step 24, the system is able to integrate the collective data and interpret the volumetric data to assess a patient's local tissue density adjacent to the tip of and outside the directional vector of the surgical tool. In other words, this step involves assessing the tissue density adjacent to the tip of and outside the directional vector of a surgical tool in preparation for presenting surgeon with feedback on such tissue density, using data collected from steps 21 and 22, and processing and interpreting the same. In other words, after the assessing step 24, the interpreted adjacent tissue density data can be presented to the surgeon to allow him or her to decide on the next surgical steps.

In one embodiment, while the processing step 23 and the assessing step 24 are shown as separate steps and in series, it will be appreciated that these steps can be performed concurrently by the same robotic system. In another embodiment, these steps may be integrated into a single step. In yet another embodiment, these steps may be provided in real-time to the surgeon during the actual surgical procedure.

Next, in step 25, the system is able to provide haptic feedback during the actual surgical procedure. The haptic feedback may include tactile detectors and other suitable haptic elements embedded throughout the robotic system. For example, a controller (not shown) for the robotic system may direct a surgeon's hand away from a vulnerable structure by providing haptic feedback to the surgeon, or otherwise, indicating the expected proximity of the vulnerable structure.

In one embodiment, the robotic system is able to present the surgeon with feedback on tissue density adjacent to the tip of and outside the directional vector of a tool to help surgeon execute tasks involving tissue manipulation.

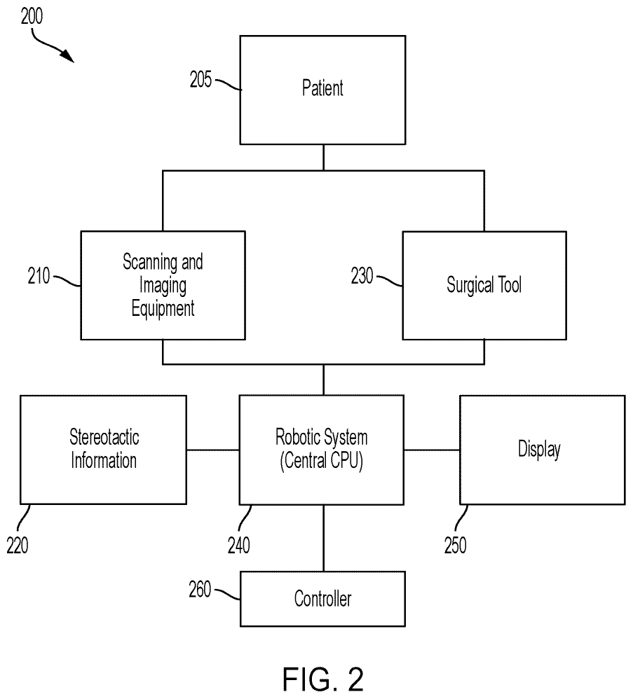

Reference is now made to FIG. 2 illustrating a surgical navigation and planning system 200 and associated tools therein according to an embodiment. In one embodiment, the system 200 includes a patient 205 and a variety of scanning and imaging equipment 210. In operation, the scanning and imaging equipment 210 can be used on the patient 205 during pre-operative procedures, or during the actual operation (e.g., intra-operative), or both. The various scanning and imaging equipment 210 can be used for CT or MRI scanning on the patient 250, among other imaging techniques, to generate the 3D imaging data related to the patient as described above. The 3D imaging data may be internally processed by computer systems integrated with the scanning and imaging equipment 210. Alternatively, the 3D imaging data may be provided to and processed by a central processing unit (CPU) such as the likes of a computer system within a robotic system 240. In another embodiment, the 3D imaging data may be provided to and processed by a separate computer system with a CPU, the separate computer system being a different computer system from the robotic system 240.

In one embodiment, the system 200 includes surgical tools 230 that are in communication with the robotic system 240. In operation, the surgical tools 230 are used to perform the relevant surgeries on the patient 205, with instructions from the robotic system 240 as dictated by the surgeon via controllers 260.

In one embodiment, the robotic system 240 and the surgical tools 230 may be integrated within a single system (e.g., all components reside within the operating room). In one embodiment, the robotic system 240 and the surgical tools 230 may be located in separate places but nevertheless maintain electronic communication (e.g., information can be wirelessly communicated).

In one embodiment, the system 200 includes stereotactic information 220 collected by the surgical tools 230 in combination with the robotic system 240 similar to those discussed above. While the stereotactic information 220 is illustrated to reside adjacent the robotic system 240, it will be appreciated that the stereotactic information 220 may be integrated within the robotic system 240. Alternatively, the stereotactic information 220 may be communicated from the surgical tool 230 to be processed by and stored within the robotic system 240. As discussed above, the stereotactic information 220 can be projected onto a display 250, which is made available to the surgeon during the surgery. In addition, the display 250 may also provide information related to the surgical tools 230 during the actual surgical procedure of the patient 205, with all relevant information relayed through and processed by the robotic system 240 or a separate computer system that is in communication with all the relevant components.

In one embodiment, haptic feedback elements similar to those discussed above may be integrated with the surgical tools 230 or the robotic system 240. The haptic feedback can provide information, throughout the surgery, as encountered by the surgical tools 230, processed by the robotic system 240, communicated to the controller 260, and ultimately relayed to the surgeon, such haptic information being optionally viewable on the display 250. In one embodiment, similar haptic feedback elements may be integrated with the controller 260 so that such tactile feedback may be experienced by the surgeon operating the surgical controller 260.

In one embodiment, the system 200 includes at least one controller 260 that is controlled and operated by the surgeon in performing the surgery. The controller 260 may include levers, buttons, consoles, among other known components for purposes of operating the surgical tools 230 (e.g., bending or rotating the surgical instruments). In some instances, the controller 260 may be integrated with the surgical tools 230 (e.g., the tool may be a scalpel at the end of a robotic arm and operated by a handle at the opposite end of the robotic arm). In other instances, the surgical tool 230, the robotic system 240 and the controller 260 may be integrated as a single unit such that all of these components are in communication with each other.

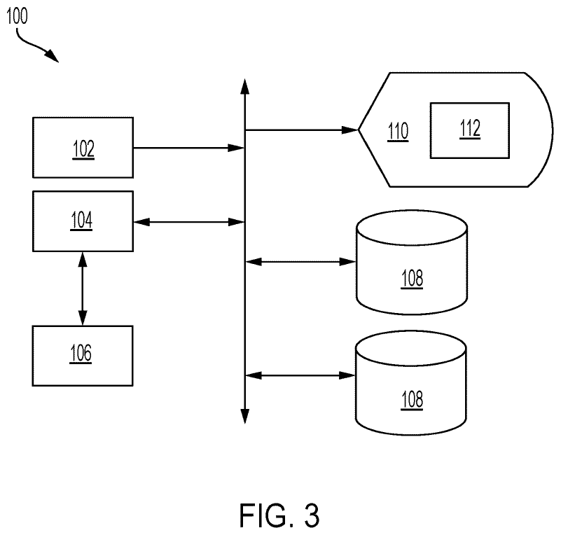

Reference is now made to FIG. 3, which illustrates an exemplary system architecture 100 for implementing the surgical navigation and planning tools, systems and methods according to the present disclosure. The system architecture 100 can include at least one processor 104 (e.g., microprocessor) communicatively coupled to an input device 102, a memory 106 or non-transitory computer readable storage medium, at least one database or data storage device 108, and a display 110 having a graphical user interface (GUI) 112. The processor 104 can be configured to execute computer readable instructions stored in the memory 106.

In one embodiment, the scanning and imaging equipment 210 as described above can be performed in a system architecture 100 as described above. For example, the CT or MRI scanning equipment may include a computer system having a processor 104 with memory 106 and database or data storage 108. The acquisition of the 3D imaging data from the CT or MRI scans can be projected on a display 110 with a GUI 112. The input device 102 may be an x-ray source (for CT) or powerful magnetics used for generating the magnetic fields (for MRI), such input device 102 in communication with suitable processor 104 for executing computer software programs that may be stored in the memory 106 with the 3D imaging data stored in the database or data storage 108.

In one embodiment, the robotic system 240 as described above can be performed in a system architecture 100 as described above. For example, the robotic system 240 may include a computer system having a processor 104 with memory 106 and database or data storage 108. The acquisition of the 3D imaging data from the scanning and imaging equipment 210 can be received within the database or data storage 108 of the robotic system 240 to be processed by the processor 104, for display on a display 110 with a GUI 112. Optionally, the display 110 of the robotic system 240 may be a standalone display 110 or may be integrated with the display 250 as discussed above. The input device 102 for the robotic system 240 may be both the scanning and imaging equipment 210 as well as the surgical tools 230, such input device 102 in communication with the suitable processor 104 for executing computer software programs that may be stored in the memory 106 for processing data stored within the database or data storage 108.

For example, the information stored within the database or data storage 108 of the robotic system 240 may include pre-operative cross-sectional imaging data from the scanning and imaging equipment 210, as well as stereotactic information 220 intraoperatively obtained from the surgical tools 230, both sets of information being collectively processed and analyzed via the processor 104 to help surgeons more safely and deftly perform surgical procedures as described above.

In one embodiment, the input device 102 for the robotic system 240 may also include the controller 260, which can allow the surgeon to view the information on the display 250 and operate the surgical tools 230 in operation of the patient 205.

In one embodiment, the robotic system 240 may further include input and output devices such as the haptic feedback elements described above. For example, haptic feedback may be received from the surgical tool 230 and communicated to the controller 260 via the robotic system 240 to be experienced by the surgeon during the surgical procedure. Alternatively, haptic feedback may be received from the surgical tool 230 for projection on the display 250 via the robotic system 240 so the surgeon can navigate the tissue densities adjacent the surgical site.

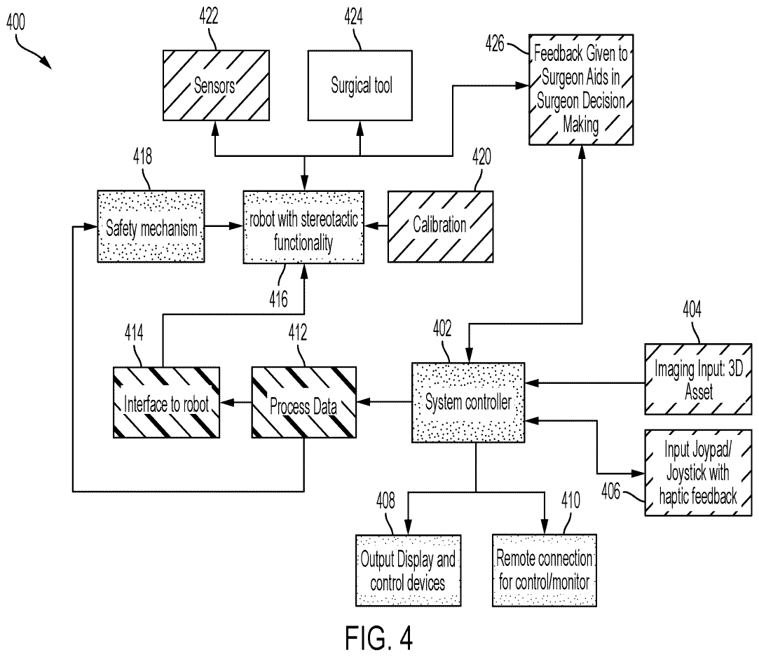

As shown in FIG. 4, an exemplary system 400 includes a system Controller 402 that interfaces with a robot and other sensors which are part of the robot and input and output devices.

System 400 includes Imaging Input—3D Asset 404. The 3D imaging data is acquired from either real-time or pre-processed data of MRI/CT scan and serves as an input to the system controller 402.

System 400 includes Input Joypad/Joystick with haptic feedback 406. This is the input that comes from the any joystick/joypad that interfaces with the system controller 402; the system controller 4-2 also sends feedback data to the joystick/joypad 406 for haptic feedback.

System 400 includes output display and control devices 408. This is the display for the system controller 402 that is used to configure and setup the entire system. The display can be touch and/or a combination of input devices with a display.

System 400 includes a Remote connection for control/monitor 410; this is the connection for use to do remote operation.

System 400 includes a processing unit 412 that processes incoming data and sends it to the robot interface and safety mechanism. This block can be a part of the system controller or a standalone unit.

System 400 includes an Interface to the robot 414 through a physical or a wireless connection.

System 400 includes a robot with stereotactic functionality 416 that provides users with an ability to perform surgeries with greater speed and increased accuracy. The electronics allied to the micro-controllers helps reduce the risk of human error and increase surgical accuracy

System 400 includes a Safety mechanism 418 that is an independent supervisory fail-safe system. It receives feedback from processed data (Imaging Input: 3D Asset) and surgical tool (Power monitoring. Etc.). The safety mechanism 418 performs anomaly detection and prevention by halting the robot operation and alerting the user.

System 400 includes a calibration unit 420 that provides the robot a real world frame of reference.

System 400 incudes sensors 422 that are interfaced with the robot to get actual positional information.

System 400 includes a surgical tool 424 that is the actual tool used to perform surgery.

System 400 includes a feedback unit 426 that generates feedback to the surgeon. The feedback is from the surgical tool and positional data from the sensors and is provided to the controller 402.

While example embodiments have been particularly shown and described, it will be understood by those skilled in the art that various changes in form and details may be made therein without departing from the scope of the embodiments encompassed by the appended claims. For example, other useful implementations could be achieved if steps of the disclosed techniques were performed in a different order and/or if components in the disclosed systems were combined in a different manner and/or replaced or supplemented by other components. In general, any combination of disclosed features, components and methods described herein is possible. For instance, various steps within a disclosed method can be performed in any order that is physically possible. Accordingly, other implementations are within the scope of the disclosure and while different embodiments have been disclosed, the invention is not limited thereby.

What is claimed is:

1. A method of using surgical navigation and planning tools, the method comprising:
obtaining three dimensional (3D) imaging data of a patient by an imaging technique;
concurrently with obtaining the 3D imaging data, obtaining stereotactic information from an intraoperative tool tracked by a surgical robotic device and associated robotic system;
processing the 3D imaging data and the stereotactic information;
integrating the 3D imaging data and the stereotactic information and interpreting volumetric data to assess a patient's local tissue density at the tip of a directional vector of the surgical tool; and
providing haptic feedback during the surgical procedure.

2. The method of claim 1 wherein the imaging technique is a magnetic resonance imaging (MRI) scan or a computerized tomography (CT) scan.

3. The method of claim 1 wherein the 3D imaging data include pre-operative data and/or intra-operative data.

4. The method of claim 1 wherein the robotic system operates concurrently with the surgical robotic device and the intraoperative tool in real-time during a surgical procedure.

5. The method of claim 4 wherein the surgical robotic device is configured to identify, confirm, and project a location of the intraoperative tool within a patient's body onto a display in real-time.

6. The method of claim 1 wherein the haptic feedback includes tactile detectors and other haptic elements embedded throughout the robotic system.

7. A system comprising:
a controller configured to receive three dimensional (3D) imaging input and feedback and generate output, the 3D imaging input comprising real-time or pre-processed data of a magnetic resonance imaging (MRI)/computerized tomography (CT) scan, the controller interfacing with a stereotactic robot.

8. The system of claim 7 further comprising a stereotactic robot device linked to the controller.

9. The system of claim 8 wherein the stereotactic robot device provides users with an ability to perform surgeries with greater speed and increased accuracy.

10. The system of claim 9 wherein the stereotactic robot device is linked to sensors to receive actual positional information.

11. The system of claim 10 wherein the stereotactic robot device is linked to a surgical tool.

12. The system of claim 11 wherein the surgical tool and the sensors are configured to send data to the controller.

13. The system of claim 12 further comprising a safety unit configured to perform anomaly detection and prevention by halting robot operation and alerting a user.

14. The system of claim 8 wherein the stereotactic robot device is linked to a calibration device configured to provide the stereotactic robot device its real world frame of reference.
